(19) United States
(12) Patent Application Publication
(10) Pub. No.: US 2005/0176677 A1
(43) Pub. Date: Aug. 11, 2005

(54) Cosmetic composition comprising monosaccharides or polysaccharides, uses and treatment methods

(76) Inventors: Claude Dal Farra, Opio (FR); Nouha Domloge, Valbonne (FR); Dominque Peyronel, Marseille (FR)

Correspondence Address: YOUNG & THOMPSON, 745 SOUTH 23RD STREET, 2ND FLOOR, ARLINGTON, VA, 22202, US

(21) Appl. No.: 10/513,025

(22) PCT Filed: Apr. 29, 2003
(86) PCT No.: PCT/FR03/01348

(30) Foreign Application Priority Data
Apr. 30, 2002 (FR) 02/05407
Apr. 30, 2002 (FR) 02/05408

(51) Int. Cl. A61K 31/732; A61K 7/06; A61K 7/11

(52) U.S. Cl. 514/54; 424/70.13

(57) ABSTRACT

A cosmetic and/or dermatological composition includes comprising, as an active ingredient, at least one mono or polysaccharide, constitutive of a pectic substance coming from a plant of the Lemnaceae family. The invention includes also its use, inter alias, for the care of the skin and/or its appendages, its use in order to treat manifestations of cutaneous ageing as well as the manifestations of inflammation and cutaneous irritation. The invention relates also to the cosmetic process of treatment of the skin using the aforementioned composition.

The present invention relates to the cosmetic and dermatological fields, and has as an aim a cosmetic and/or dermatological composition comprising, as an active ingredient, at least one mono or polysaccharide, constitutive of a pectic substance coming from a plant of the Lemnaceae family.

The plants of the Lemnaceae family are monocotyledon angiosperm plants. The Lemnaceae family belongs to the order of Arales and regroups four genus: Lemna, Spirodela, Wolffia and Wolffiela, representing about up to thirty species in the world. Five of them belong to the European flora: Lemna triscula, Lemna minor, Lemna gibba, Spirodella polyrrhiza, Wolffia arrhiza.

The plants of the Lemnaceae family are commonly called “duckweeds” or minor duckweed. They are small aquatic perennial plants, floating on surface of water or sometimes between two waters, without stems nor leaves, reduced to a simple horizontal lenticular corpuscle and rounded, called frond. They have one or more filiform roots, not ramified and without absorbing hair, playing a role of anchoring.

Duckweeds have various uses, they are sometimes produced for food use; collected and dried, they are sold in certain dietetic stores. In certain developing countries, duckweeds are used in animal food, such as poultry or fish food. Their success is due to the very high protein content; indeed, proteins account for 35 to 50% of dry weight, that is to say as much as the protein quantity present in Soya beans. Moreover, duckweeds have other determining advantages: by their very fast growth, they contribute to cleanse stagnant waters; they are thus used effectively as regards decontamination.

The plants of the Lemnaceae family (as all plants) present the characteristic to contain particular polysaccharides composing the structure of the vegetable cell walls: pectins. The pectins of duckweeds are particular, notably, in their composition. These are called apiogalactoronan or lemnan, with regard to the pectins of the plants species Lemna minor.

The pectic substances, or pectins, are macromolecule of a glucidic nature, essentially composed of galacturonic acids.

The monomeric motif for the pectic substances is the α D-galaturonic acid, the skeleton of the pectic substances is thus formed by dependent galacturonic acids α [1-4] linked, this chain constituting the polygalacturonic acid or pectic acid. In general, pectic substances have a more complex structure resulting from the substitution of certain groups on the principal chain.

Duckweeds have a particular pectic substance: the apiogalactoronan. The pectic substance of the duckweed Lemna minor is ramified. This apiogalactoronan was isolated from the duckweed and was characterized for the first time by Hart and Kindel in 1970.

The composition of the apiogalactoronan is particular. Indeed, the saccharidic chain of the pectic substance of Lemna minor (or lemnan) is composed of D-galacturonic acid (64%) and of D-apiose (25%) as principal components, as well as galactose, arabinose, rhamnose and xylose.

Apiose is not a very common sugar, it is a pentose with ramified chains. It was first described by Bell et al. (1954) as a component of polysaccharides of Posidonia australis.

It is known to use pectic substances in many fields, particularly in certain therapeutic fields where they are used as a thickening agent or as excipient.

However a use of saccharides, constituents of a pectic substance coming from a plant of the Lemnaceae family, as an active ingredient, in the cosmetic and/or pharmaceutical field is not known yet.

However, the inventors of the present invention have unexpectedly and surprisingly noted that the mono- or polysaccharides constituents of a pectic substance, coming from a plant of the Lemnaceae family, present specific biological activities, which make them directly usable in cosmetic, dermatological and pharmaceutical preparations or compositions to external topic use.

Thus, the main object of this invention consists of a cosmetic and/or dermatological composition comprising, as an active ingredient, at least a mono- or polysaccharide, constituent of a pectic substance coming from a plant of Lemnaceae family.

The inventors noted in particular that these saccharides present anti-inflammatory, calming, hydrating, protective and significant anti-aging actions within the framework of the above mentioned use. Moreover, these saccharides present an anti-hyaluronidase effect, that is to say that they inhibit the activity of the hyaluronidases, enzymes which hydrolyze the hyaluronic acid.

Preferentially, the composition according to the invention will comprise, as an active ingredient, the pectin contained in the plants of the Lemnaceae family.

According to a particular way of realization of the invention, the polysaccharide coming from Lemnaceae family is a pectic substance comprising at least an apiose motif.

According to another way of realization of the invention, the composition according to the invention will comprise, as an active ingredient, a monosaccharide, the apiose, constituent of a pectic substance coming from Lemnaceae family.

Moreover, according to another way of realization of the invention, the plants of the Lemnaceae family are plants of the Lemna genus. Of course, the pectic substances can be obtained from at least any of the many varieties and species belonging to the Lemna genus.

Preferentially, the pectic substances are obtained from at least a plant of the Lemna minor species.

According to the invention, the term “active ingredient” can be defined as being all the molecules or all unit of molecules likely to bring modifications or modulations to the operation of a biological system.

Active compounds according to the invention, that is the mono- or polysaccharides constituents of the pectic substance, are obtained from pectin. The mono- or polysaccharides constituents of a pectic substance mean all pectic substances as well as all saccharides or saccharidic chains which compose them. The mono- or polysaccharides constituents of a pectic substance thus indicate the pectins which were more or less hydrolyzed.

An essential characteristic of the invention is that these pectic substances, from which are drawn the mono- or polysaccharides, come from a plant of the Lemnaceae family.

Thus, any method of extraction and/or purification known by the person skilled in the art can be used to prepare the glucidic compounds according to the invention.

Of course, the effective amount of active ingredient, constituting the composition, corresponds to the necessary amount in order to obtain the desired result.

According to an advantageous way of realization of the invention, the mono- or polysaccharide, constituent of a pectic substance coming from a plant of the Lemnaceae family, is present in a quantity representing from 10⁻⁵% to 20% of the total weight of the composition and, preferentially, in a quantity representing from 10⁻⁴% to 5% of the total weight of the composition.

According to an advantageous way of realization of the invention, the active compounds above mentioned are beforehand solubilized or dispersed in one or more cosmetically or pharmaceutically acceptable solvents such as water, ethanol, propanol or isopropanol, propylene glycol, butylene glycol, dipropylene glycol, diglycols ethoxylated or propoxylated, cyclic polyols, petroleum jelly, a vegetable oil or any combinations of these solvents.

According to another advantageous way of realization of the invention, the above mentioned active compounds are beforehand solubilized in a cosmetic or pharmaceutical vector such as liposomes or are adsorbed on powdery organic polymers, mineral supports such as talc and bentonites, and more generally solubilized in, or fixed on, any cosmetically or pharmaceutically acceptable vector.

The composition, according to the present invention, can be a cosmetic or dermatological or pharmaceutical composition. According to this invention, the composition is preferably cosmetic in nature, since it is aimed at improving the cutaneous appearance and general cutaneous performance of the individual who uses it.

According to the present invention, the composition is preferably a cosmetic and/or dermatological composition adapted for topical cutaneous application through an acceptable cosmetic or dermatological medium.

It is evident that the invention relates to mammals in general, and more specifically, to humans. Whatever forms the invention takes, the composition, according to the invention, can be ingested, injected, or, applied to skin (to all cutaneous zones of the body), hair, nails or mucous membranes. According to the mode of administration, the composition related to the invention can be presented under all galenic forms normally used.

The compositions related to the invention are preferably presented under a galenic form adapted for cutaneous topical administration. They cover all the cosmetic and dermatological forms. These compositions must contain an acceptable cosmetic or dermatological medium. That is to say, a medium that is compatible with skin and hair.

These compositions can take the form of an aqueous, hydro-alcoholic or oil solution; or the form of an oil-in-water, water-in-oil emulsions or in multiple emulsions. They can also be used as creams, as suspension or as a powder, adapted for application to skin, mucous membranes, lips and/or hair.

These compositions can also be more or less fluid or solid and can take the form of creams, lotions, milks, serums, ointments, shampoo, gel, paste and mousse. It can also take a solid form like a stick, or it can be used, on the skin, in aerosols. It can also be used as a skin care product and/or as make-up for skin.

Concerning injection, the composition related to the invention can be aqueous or oil based lotions or a serum. For application to the eyes, the composition can be used as drops whereas for ingestion it can be used as capsules, granules, syrup or pills.

Moreover, these compositions can include all of the additives that are usually considered for use in this application as all the possible additives necessary for their formulation such as solvents, thickeners, diluents, anti-oxidants, colorants, solar filters, auto-tanning products, pigments, fillers, preservatives, perfumes, odor absorbers, pharmaceutical and cosmetic active ingredients, essential oils, vitamins, essential fatty acids, tensioactivators, filmogen polymers etc.

In all the cases, the person skilled in the art will attempt to carefully consider the selection of adjuvants, as well as their proportions, not to compromise the advantageous properties of the composition relating to the invention. These adjuvants can, for example, correspond to 0.01% to 20% of the total weight of the composition.

When the composition according to the invention is an emulsion, the fatty phase can represent 5% to 80% by weight, but preferably it would represent 5% to 50% of the weight with respect to the total weight of the composition. Emulsifiers or co-emulsifiers used in the composition are selected among those that are classically used in the domain under consideration. For example, they can be used in a proportion of 0.3% to 30% by weight relative to the total weight of the composition.

Of course, the person skilled in the art should select the complementary compounds for the composition, active or non-active, as well as the amounts of the complementary compounds in such a way that the advantageous properties of the composition will not be perceptibly altered by the envisaged addition.

The compositions, according to the present invention, can be applied most notably as a cosmetic or pharmaceutical composition for use on the skin, mucous membranes and/or semi-mucous membranes.

The compositions can be applied, particularly, as a skin protection and/or skin care products, or as an anti-wrinkle and/or anti-aging composition.

We can also consider an application in the field of facial and body make-up compositions, such as lipsticks, foundation, tinted creams, dark circle sticks, or sunscreen and artificial tanning compositions.

The compositions of the invention can be used in a great number of treatments, notably cosmetic and dermatological treatments. They can take the form of cosmetic compositions used for skin, lips and/or hair treatment, protection, care and make-up removal and/or cleaning, as well as for make-up applications to skin, lips, eye lashes and/or the body.

The composition relating to the invention can also consist of solid preparations such as soap and other cleaning bar soaps.

The composition can also be made in aerosol form in which it can be mixed with pressurized propulsion agents.

The composition can also be used orally, for example, as toothpaste. The composition of the invention can be applied as a cosmetic, dermatological or pharmaceutical composition to be used orally. These can take the form of drinkable solution, syrups, tablets, sugar coated pills, capsules, or even as food and nutritional supplements.

According to the invention, we can add to the composition of the invention, other active agents intended for the prevention and/or treatment of the manifestations of cutaneous aging, and/or skin protection from external aggressions.

The invention has, as well, for object the use, as an active ingredient, of at least a mono- or polysaccharide, constituent of a pectic substance coming from a plant of the Lemnaceae family, such as previously defined, in or for the preparation of a composition; the compounds or the composition comprising it are used in order to fight in a curative and/or preventive way, against the signs of cutaneous aging, but also in order to improve the aspect of the skin and its appendages. In a more general manner, the composition according to the invention will have an activity in the care of the skin and its appendages.

The care of the skin and/or its skin appendages is the process of conserving or re-establishing the healthy functioning of skin and/or its skin appendages or the process of providing the means that serve to preserve or improve their appearance and/or texture.

Thus, skin care refers to hydration, appeasement, and protection against all types of aggression, especially from the sun, as well as the fight to prevent the manifestations of aging, particularly cutaneous manifestations of aging.

Manifestations of cutaneous aging include all the modifications regarding external appearance of skin due to aging. Examples of these modifications include wrinkles and fine lines, limp skin, slackened skin, slimmer looking skin, loss of elasticity and/or skin tone, dull skin, and skin which lacks radiance. It also includes internal skin modifications that do not translate directly into changes in external skin appearance. An example of these internal modifications is the degradation that occurs internally in skin resulting from consecutive exposure to UV radiation.

The phrase “to improve the appearance of the skin” refers to all the phenomena which are likely to have for consequences a visual improvement of the state of the skin. The skin will have a better appearance; it will be, for example, much more beautiful, firm and/or smooth. All the small imperfections will be decreased or removed. The papyraceous aspect of the skin, for example, will be attenuated.

In addition, according to an essential characteristic, the invention relates to the use of at least a mono- or polysaccharide, constituent of a pectic substance coming from a plant of the Lemnaceae family, in order to maintain and/or to stimulate the cutaneous hydration and/or in order to fight against the drying effect on the skin.

Indeed, the mono- or polysaccharides, constituents of a pectic substance coming from a plant of the Lemnaceae family, have an anti-hyaluronidase action. That is they inhibit the activity of the enzymes, the hyaluronidases, which degrade the hyaluronic acid. However, the hyaluronic acid is the main glycosaminoglycan of the dermis and it exerts a very important role. It is indeed known that skin aging, as well as the drying effect observed in particular in old skins is, for a very significant part, related to a loss of hyaluronic acid.

It was observed that the mono- or polysaccharides, constituents of a pectic substance coming from a plant of the Lemnaceae family, according to the invention, has many actions in the skin; in particular, it is possible to fight against the cutaneous aging phenomena and enables the skin to be protected against all types of external aggressions. Thus, the composition according to the invention can be used to protect keratinous substrates and, particularly, the skin and its appendages from all types of external aggression.

The use of these compounds, or the composition including it, will allow the keratinous substrates to be protected and to better resist stress produced on them by the environment. The phrase “external aggressions” refers to the aggressions which the environment can produce. These aggressions can be of chemical, physical, biological or thermal origin. As an example, we can quote aggressions such as pollution, UV, frictions, water with strong limestone concentration, variations in temperature or potentially irritating products such as tensioactives, preservatives or fragrances.

The mono- or polysaccharides, constituents of a pectic substance coming from a plant of the Lemnaceae family, have an effective action on the level of the cutaneous barrier. So, these compounds reinforce the barrier function of the skin. They promote, thus, tissue regeneration. So the compounds or the composition according to the invention can be used to accelerate tissue regeneration and/or skin wound healing.

In addition, the invention also relates to the use as an active ingredient, of the mono- or polysaccharides, constituents of a pectic substance coming from a plant of the Lemnaceae family, in or for the preparation of a composition, the compounds or the composition being intended to treat the appearance of inflammation and cutaneous irritation. The composition, indeed, is very well adapted to sensitive and delicate skins.

The compounds or the composition have a particularly effective action on the fight against allergic phenomena, and in particular on their consequences to the skin. Thus, another aspect of the invention, is the use of the compounds mentioned above, or the composition containing them, in order to fight allergic phenomena and/or the appearance of cutaneous inflammation.

In addition, the compounds or the composition, according to the invention, have a particularly effective action on the fight against the microbial agents. The compounds or the composition containing it, thus have antimicrobial properties, that is anti-bacterial, anti-fungic, virucides or anti-protozoa properties. The composition will thus have a particularly useful application in the treatment of cutaneous infections, of inflammatory phenomena of the skin and/or the hair, also in all hair and/or skin diseases having a microbial origin. More particularly, the composition according to the invention will have effectiveness in the treatment of the skin and in the fight against the bacteria. The compounds or the composition according to the invention will thus have a bacteriostatic effect.

Thus, the compounds or the composition containing it, according to the invention, can be used in the treatment of the local troubles on the skin. Moreover, they will make it possible to fight, in a particularly effective manner, acne phenomena and/or all sorts of infections or inflammatory reactions.

Another characteristic of the composition according to the invention, is its effective action in cell detoxification, i.e. the compounds or composition containing it will be useful to detoxify cells, and particularly, skin's cells.

These various properties can be used to carry out compositions intended to protect skin against external aggressions caused, in particular, by solar radiation or by other physical, chemical or biological agents; or to carry out compositions able to fight skin aging, and to care of it.

According to another aspect, the present invention relates to a cosmetic process of treatment of the skin, particularly old skins, and/or to fight cellular aging phenomena consisting in applying to the surface of the skin an effective amount of the composition such as previously defined, i.e. containing at least a mono- or polysaccharide, constituent of a pectic substance coming from a plant of the Lemnaceae family, to obtain the desired effect.

The present invention relates, in the same way, to a cosmetic process of treatment in order to protect the skin and its appendages against all types of external aggression, and/or to reinforce the barrier function of the skin and its appendages.

Another aspect related to the present invention concerns a cosmetic process for the treatment of skin in order to favor skin wound healing and tissue regeneration, but also in order to favor cellular differentiation.

The present invention also relates to a cosmetic process of treatment in order to treat allergic and/or inflammation phenomena of the skin; also in order to fight against microbial agents, consisting in applying to the surface of the skin and/or its appendages, an effective amount of the composition such as previously defined.

Particular modes of realization of this cosmetic process of treatment also result from the preceding description.

The process of cosmetic treatment related to the invention can be implemented, particularly, when applying the cosmetic compositions here above according to methods habitually used for compositions, such as the application of creams, gels, serums, lotions, milks, shampoo, and sun creams, on skin, hair and as a toothpaste applied to the gums.

Other advantages and characteristics of the invention will become apparent by reading the following examples, by way of an illustrative and unrestrictive demonstration of data.

### EXAMPLE 1

### Preparation of an Extract Containing Mono- or Polysaccharides, Constituents of a Pectic Substance, Coming From a Plant of the Family Lemnaceae

Duckweeds, of the Lemna genus, are collected on the surface of water, then washed in very pure water. 5 kg of these freshly collected, or preserved by freezing, are crushed very finely and homogenized, using an ultra-turrax blender, in 2 to 5 volumes of a saline solution, preferably in a sodium chloride solution. This operation is carried out at a temperature between 4 and 90° C., preferably at a temperature approximately equal to 40° C.

Then the crushed extract undergoes a stage of filtration and/or centrifugation; the filtrate is eliminated. This extraction operation in saline solution, followed by a stage of centrifugation, is carried out three times, each time the filtrates are isolated. The residue is recuperated and is treated in the same manner, by extraction under agitation then by centrifugation and/or filtration, three times, in water. Then a crushed extract is obtained containing the cell walls of the duckweeds. According to an alternative of the process of extraction, this stage can be carried out in the presence of enzymes, preferably of protease and/or cellulase type.

The pectin extraction, present in the cell walls of the duckweeds, is performed in aqueous solution containing a chelating agent, the ammonium oxalate, with a concentration between 0.02 and 0.1 M (EDTA or sodium polyphosphate can also be used). This extraction is realized in acidic conditions, with a pH between 2 and 5 (reached with the use of hydrochloric acid), at a temperature between 4 and 90° C.

Pectins thus extracted are precipitated by salts or alcohol. They are then collected and dialyzed to be purified. Pectins obtained can be put in aqueous solution, constituting a first usable form of the extract according to the invention.

Pectins obtained can be used in this native form or in a more or less hydrolyzed form, that is a more or less depolymerized form. In this case, pectin hydrolysis can be carried out by various pectolitic enzymes; this hydrolysis can also be carried out in a chemical way, preferably in acid medium.

The hydrolysis involves the formation of residues of, more or less, low molecular weights; thus we obtain polysaccharide, constituents of a pectic substance, of various sizes.

A total hydrolysis will release all the pectic components, and will allow a mix, mainly made up, of monosaccharides, mainly containing galacturonic acid and apiose.

The various depolymerized pectin compounds, more or less purified, constitute forms of extract usable to prepare the composition according to the invention.

According to a particular mode of realization of the invention, pectin will be completely hydrolyzed so as to obtain monosaccharide constituents of a pectic substance. This hydrolysis, followed by a stage of purification, will allow a solution containing, almost exclusively, apiose.

This solution will constitute a form of extract directly usable to carry out a cosmetic composition containing apiose.

### EXAMPLE 2

### Preparation of Compositions

These compositions were obtained by simple combination of the various components. The quantities indicated are expressed by percentage weight.

1 - Emulsion oil in water

| | |
|---|---|
| Oily phase: | |
| Montanov 68 (Cetearyl Alcohol and Cetearyl Glucoside) | 5.00% |
| Oil of Jojoba | 5.00% |
| Oil Petroleum jelly | 5.00% |
| Isopropyl Palmitate | 7.00% |
| Aqueous phase: | |
| Glycerin | 5.00% |
| Allantoïne | 0.10% |
| extract of example 1 | 5.00% |
| Sepigel 305 (Polyacrylamide, C13-14 Isoparaffin and | 0.30% |
| Laureth-7) | |
| Conservative | 0.50% |
| Perfume | 0.50% |
| Water | qsp 100% |

2 - Freezing

| | |
|---|---|
| Carbopol Ultrez 10 (ground with 2%) | 25% |
| Triethanolamine | 0.50% |
| Extract of example 1 | 5.00% |
| Conservative | 0.20% |
| EDTA (sequestering) | 0.10% |
| Perfume | 0.50% |
| Water | qsp 100% |

3 - Lotion

| | |
|---|---|
| Mono Propylene Glycol | 1.00% |
| Allantoïne | 0.30% |
| Glycerin | 1.00% |
| Cetiol HE (Peg-7 Glyceryl Cocoate) | 1.00% |
| Extract of example 1 | 5.00% |
| Conservative | 0.20% |
| Perfume | 0.50% |
| Water | qsp 100% |

1. Cosmetic and/or dermatological composition comprising, as an active ingredient, in an physiologically acceptable medium, an effective amount of at least a mono- or polysaccharide, constituent of a pectic substance coming from a plant of the Lemnaceae family.

2. Composition according to claim 1 characterized in that the polysaccharide, coming from a plant of the Lemnaceae family, is a pectic substance.

3. Composition according to claim 1 characterized in that the polysaccharide coming from the Lemnaceae family is a pectic substance comprising at least an apiose motif.

4. Composition according to claim 1 characterized in that the monosaccharide, constituent of a pectic substance coming from a plant of the Lemnaceae family, is the apiose.

5. Composition according to claim 1 characterized in that the plants of the Lemnaceae family are of Lemna genus.

6. Composition according to claim 5 characterized in that the plants belong to the Lemna minor species.

7. Composition according to claim 1 characterized in that the mono- or polysaccharide, constituent of a pectic substance coming from a plant of the Lemnaceae family, is present in a quantity representing from 10⁻⁵% to 20% of the total weight of the composition and, preferentially, in a quantity representing from 10⁻⁴% to 5% of the total weight of the composition.

8. Composition according to claim 1 characterized in that it takes a cosmetic and/or dermatological form adapted to topical cutaneous administration for the skin, mucous membranes and/or the skin's appendages, comprising a pharmaceutically or cosmetically acceptable medium.

9. Composition according to claim 1 characterized in that the active compounds are beforehand solubilized or dispersed in one or more cosmetically or pharmaceutically acceptable solvents such as water, ethanol, propanol or isopropanol, propylene glycol, butylene glycol, dipropylene glycol, diglycols ethoxylated or propoxylated, cyclic polyols, petroleum jelly, a vegetable oil or any combinations of these solvents.

10. Composition according to claim 1 characterized in that the active compounds are beforehand solubilized in a cosmetic or pharmaceutical vector, such as liposomes, or are adsorbed on powdery organic polymers, mineral supports such as talc and bentonites, and more generally solubilized in, or fixed on, any cosmetically or pharmaceutically acceptable vector.

11. Composition according to claim 1 characterized in that it takes the form of an aqueous, hydro-alcoholic or oil solution; or the form of an oil-in-water, water-in-oil emulsions or in multiple emulsions; or the form of creams, suspension or powder; these compositions can be more or less fluid or solid, and take the form of a cream, a lotion, a milk, a serum, an ointment, a shampoo, a gel, a paste, a mousse or a stick.

12. Use of at least a mono- or polysaccharide, constituent of a pectic substance coming from a plant of the Lemnaceae family, such as defined by claim 1, as an active ingredient, in or for the preparation of a composition; the compounds or the composition are used in order to treat, in a curative and/or preventive way, the signs of cutaneous aging, and/or to improve the appearance of the skin and/or its appendages.

13. Use of at least a mono- or polysaccharide, constituent of a pectic substance coming from a plant of the Lemnaceae family, such as defined by claim 1, as an active ingredient, in or for the preparation of a composition; the compounds or the composition are used to favor tissue regeneration and/or skin wound healing.

14. Use of at least a mono- or polysaccharide, constituent of a pectic substance coming from a plant of the Lemnaceae family, such as defined by claim 1, as an active ingredient, in or for the preparation of a composition; the compounds or the composition being intended to fight against the cutaneous allergic phenomenon.

15. Use of at least a mono- or polysaccharide, constituent of a pectic substance coming from a plant of the Lemnaceae family, such as defined by claim 1, as an active ingredient, in or for the preparation of a composition; the compounds or the composition being intended to fight against the appearance of cutaneous inflammation.

16. Use of at least a mono- or polysaccharide, constituent of a pectic substance coming from a plant of the Lemnaceae family, such as defined by claim 1, as an active ingredient, in or for the preparation of a composition; the compounds or the composition being intended to maintain and/or to stimulate the cutaneous hydration and/or in order to fight against the drying effect on skin.

17. Use of at least a mono- or polysaccharide, constituent of a pectic substance coming from a plant of the Lemnaceae family, such as defined by claim 1, as an active ingredient, in or for the preparation of a composition; the compounds or the composition being intended to detoxify the cells.

18. Use of at least a mono- or polysaccharide, constituent of a pectic substance coming from a plant of the Lemnaceae family, such as defined by claim 1, as an active ingredient, in or for the preparation of a composition; the compounds or the composition being intended to fight against microbial agents and notably against bacteria.

19. Use of at least a mono- or polysaccharide, constituent of a pectic substance coming from a plant of the Lemnaceae family, such as defined by claim 1, as an active ingredient, in or for the preparation of a composition; the compounds or the composition being intended to protect the skin and/or its appendages against external aggressions, and/or to reinforce the barrier function of the skin.

20. Use of at least a mono- or polysaccharide, constituent of a pectic substance coming from a plant of the Lemnaceae family, such as defined by claim 1, as an active ingredient, in or for the preparation of a composition; the compounds or the composition being intended for their anti-hyaluronidase action.

21. Cosmetic process of treatment in order to treat aged skins and/or to fight cellular aging phenomena, and/or to protect the skin and its appendages against external aggressions, and/or to reinforce the barrier function of the skin and its appendages, consisting in applying, to the skin's surface and/or to the appendages, an effective amount of the composition such as defined according to claim 1.

22. Cosmetic process of treatment in order to favor wound healing and/or tissue regeneration, and/or to favor cellular differentiation, consisting in applying, to the skin's surface and/or to the appendages, an effective amount of the composition such as defined according to claim 1.

23. Cosmetic process of treatment in order to treat allergic and/or inflammation phenomena, and/or in order to fight against microbial agents, consisting in applying, to the skin's surface and/or to the appendages, an effective amount of the composition such as defined according to claim 1.
